(19) United States
(12) Patent Application Publication
(10) Pub. No.: US 2020/0000710 A1
(43) Pub. Date: Jan. 2, 2020

(54) Systems Methods Devices Apparatuses Circuits and Computer Executable Code for Production and Topical Application of a Therapeutic Substance

(71) Applicant: Zion Azar, Shoham (IL)

(72) Inventor: Zion Azar, Shoham (IL)

(21) Appl. No.: 16/482,264

(22) PCT Filed: Jan. 31, 2018
(86) PCT No.: PCT/IB18/50597

§ 371 Date: Jul. 31, 2019

Related U.S. Application Data
(60) Provisional application No. 62/453,011, filed on Feb. 1, 2017.

(51) Int. Cl. A61K 9/00; A61K 31/185; A61K 47/69; A61M 31/00

(52) U.S. Cl. CPC A61K 9/0009; A61K 9/0004; A61K 9/0012; A61K 31/185; A61M 2210/04; A61M 31/002; A61M 2202/0007; A61M 2205/36; A61K 47/6943

(57) ABSTRACT

Disclosed is a therapeutic substance application packet consisting of two or more, different, size ranges of therapeutic substance granules, wherein granules of the different size ranges have different dissolving and exfoliating properties. Further disclosed are an apparatus for application of therapeutic substance application packet and a process for production of same.

### RELATED APPLICATIONS

This application claims the priority of applicant's U.S. Provisional Patent Application No. 62/453,011, filed Feb. 1, 2017. The disclosure of the above mentioned 62/453,011, Provisional patent application, is hereby incorporated by reference in its entirety for all purposes.

### FIELD OF THE INVENTION

The present invention relates generally to the field of topical therapeutic substances and their application. More specifically, the present invention relates to therapeutic compounds and mixtures, to processes for producing same, and to systems, methods, devices and apparatuses for application of the therapeutic compounds and mixtures.

### BACKGROUND

Exposure of the skin to environmental conditions, especially to UV light, generates reactive oxygen species (ROS). These radicals have a potential to start chain or cascade reactions that damage the cells. The harmful effects of ROS occur as direct chemical alterations of the cellular DNA, the cell membrane and the cellular proteins, including collagen which leads to aging skin. Antioxidants counter this natural aging process, and that's why they have been taking top billing in skin care.

Oxidation occurs throughout nature. In the case of our skin, it is largely caused by the creation of free radicals at the cellular level when skin is exposed to ultraviolet light. Antioxidants are touted to reduce oxidative stress by neutralizing free radicals—unstable atoms that have an unpaired electron in their outermost shell, almost like a knife without a sheath. The antioxidants act to sheathe the knife, binding with the unstable electron and stopping it from attacking collagen strands and other cells of the skin.

Well Known Antioxidants are compounds such as vitamins C and E, Coenzyme Q10, Idebenone, Zinc, Copper and Beta Carotene. Beauty companies are harnessing these, as well as the antioxidants from an increasing range of botanicals such as green tea, pomegranates, coffee berries, grape seeds, olives, mushrooms and more.

Major issues in using antioxidants in cosmetics are: how to keep antioxidants stable in product formulas and how well antioxidants are actually absorbed into the skin.

Stability: Since antioxidants degrade upon exposure to UV light, moisture and oxygen, some skincare companies are solving the problem of rapid antioxidant breakdown by packaging lotions, creams and serums in dark brown, blue, or opaque bottles and in metal tubes.

Absorption: Antioxidants that are taken by mouth either in food or as supplements are circulated through the body and absorbed into cells. But when it comes to applying them to the skin, the concern has been that they would just sit on top of it, where they would soon be washed or rubbed off instead of being absorbed into the skin cells where their protective action could be most effective. The upper most layer of the skin, the Stratum Corneum, is designed to reject any material from entering the skin. For example Vitamin C is available in a number of active forms. Among available options, L-ascorbic acid is the most biologically active and well-studied. L-ascorbic acid is a hydrophilic and unstable molecule, hence the poor penetration into the skin due to the hydrophobic character of the Stratum Corneum. L-ascorbic acid is also a charged molecule, which further limits its penetration into the skin.

There remains a need, in the field of topical therapeutic substances and their application, for: therapeutic and cosmetic substances; methods of production thereof; systems, apparatuses, devices and/or procedures for their application and effective penetration of treated subjects' skin; and/or tools for their (e.g. antioxidant substances) protection from degradation.

### SUMMARY OF THE INVENTION

The present invention includes therapeutic compounds and mixtures, processes for producing same; and to systems, methods, devices and apparatuses for the topical application of the therapeutic compounds and mixtures. According to some embodiments of the present invention, there may be provided a Therapeutic Substance Application Packet (TSAP), which packet may include a mixture of two or more types of therapeutic substance granules (TSG), wherein granules of a first TSG type may be of a different size than granules of a second TSG type. According to some embodiments. Granules of the first TSG type may be composed of the same or similar composition of therapeutic substances as are granules of the second TSG type. Alternatively, granules of the first TSG type may be composed of different compositions of therapeutic substances from granules of the second TSG type. A TSAP according to embodiments may include a preselected ratio of first type to second type TSGs. A size range of granules of the relatively smaller TSG type may be selected such as to dissolve relatively faster upon contact with a solvent during topical application, while a size range of granules of the relatively larger TSG type may be selected to dissolve relatively slower upon contact with a solvent during topical application and to retain exfoliating properties for at least a portion of a topical application period of the TSAP.

According to some embodiments of the present invention, a TSG may be composed of one or more medical, therapeutic, nutritional and/or cosmetic substances and/or active therapeutic substance types, including: (1) antioxidants; (2) hair growth stimulants; (3) hair removal substances; (4) skin nourishing and moisturizing substances; (5) skin protection substances; and/or (6) any combination of the above, optionally including the addition of one or more complementary substance(s), of the following substances: Vitamin A, Vitamin D, Vitamin E, Vitamin K, Vitamin C, Thiamin, Riboflavin, Niacin, Vitamin B6, Vitamin B12, Pantothenic Acid, Folic Acid, Biotin, Hyaluronic acid, Coenzyme Q10, Idebenone, Zinc, Copper, Beta carotene, Green tea, Pomegranates, Coffee berries, Grape seeds, Saw palmetto, Epilobium, Retinol, Peptides, Alpha and Beta hydroxy acids, Azelaic acid, Ursolic acid, Oleanolic acid and/or 4-Butyl Resorcinol.

According to some embodiments, the one or more TSG substance(s) may chemically react and/or dissolve, upon contact with a solvent previously applied to a treated organ section (e.g. face skin) of a subject, on which the TSAP is being applied. The solvent may be water or oil based and may take the form of: a gel, an oil, a cream or ointment, a lotion and/or a liquid. The solvent may be applied to the treated organ (e.g. face skin) of the subject, prior to the application of the TSAP and TSGs thereof, such that upon application one or more of the TSGs chemically reacts with the applied solvent, at least partially dissolving the granules of the TSG(s) to yield an infusible therapeutic fluid substance.

According to some embodiments of the present invention, a TSAP may include, in addition to the two or more TSGs, any combination of the following substances, elements, materials and/or compounds: (1) a Catalyst for increasing the rate of a chemical reaction without itself undergoing any permanent chemical change; (2) a Binder/Adhesive for connecting between two or more substance type powders (e.g. an active substance powder and a secondary substance powder) to form a solid limp which is later milled and granulated to form the TSGs; (3) a Preservative for preserving the qualities of the TSGs' active substances (e.g. antioxidants) and extending the time period during which they remain effective; (4) a Lubricant for minimizing friction and separating TSGs while they are sieved into different size ranges; (5) Breakable microcapsules of solvent, or microencapsulated solvent, for breaking, during TSAP application, and releasing their internally (i.e. within capsule) contained solvents to dissolve the TSG(s) and collectively yield/form a therapeutic liquid; (6) Desiccant/Dehumidifying substances for acting as a TSG(s) drying agent (e.g. hygroscopic substances) used to prevent moisture from: contacting, reacting with and/or decreasing the effectiveness of, the TSG(s); (7) Inert abrasive/exfoliant for exfoliating the skin of a TSAP treated organ/area of a subject (e.g. face skin), thus facilitating the infusion of dissolved TSG(s) into the skin, wherein the inert attribute prevents the abrasive/exfoliant from chemically reacting with its surrounding substances (e.g. other TSG(s), solvent) thus retaining its structure and respective exfoliating characteristics; and/or (8) Any other type of Additive.

According to embodiments of the present invention, there may be provided a TSAP Application Apparatus to receive a TSAP and to apply its contents to the skin of a treated subject. The apparatus may include any combination of the following components: (1) an Electric Power Source (e.g. a rechargeable battery) for providing electric power to digital and mechanical components of the apparatus; (2) a TSAP Holder for receiving and connecting a TSAP to the apparatus, retaining it in position during use/operation and facilitating the application of the therapeutic substances thereof; (3) an Electric Motor, powered by the power source, for generating rotational motion and transmitting the generated motion to the TSAP holder and the TSAP retained thereby; and (4) a CPU/Control-Logic for triggering and managing the operation of the electric motor and thereby facilitating the application of the therapeutic substance(s) contained in the retained TSAP onto the surface of the skin of a treated subject and/or exfoliating the skin in and/or around the treated area to assist the infusion of therapeutic substance(s).

According to some embodiments, the different components of the TSAP application apparatus may be functionally enclosed in a body/casing; according to some embodiments, the body/casing may include a handle section connected, optionally in a right or approximately right angle, to an application and TSAP holding section, such that when a user of the apparatus holds the apparatus by the handle section, the outer surface of a TSAP retained by the TSAP holder is in a substantially horizontal plane to the surface of the treated subject's skin. According to some embodiments, the body/casing may constitute the handle, or holding area, of the TSAP application apparatus.

According to some embodiments, the TSAP application apparatus may further include a control interface, having user accessible interface elements, for powering the apparatus and for operating some or all of the motors/components/unit/controllers/functionalities of the apparatus as described herein.

According to some embodiments, the rotational motion of the TSAP holder and the TSAP retained thereby, may be in a substantially horizontal plane to the surface of the subject's skin, to enhance the application and the dissolving of the therapeutic substances onto the surface of the skin of the subject, by increasing the amount of contact between the TSAP granules and a solvent previously applied to the treated skin area of the subject.

According to some embodiments, the rotational motion of the TSAP over the treated subject's skin may, or may mostly, enhance the application and dissolving of the size range of granules of the relatively smaller TSG type, which dissolve relatively faster upon contact with a solvent during topical application.

According to some embodiments, the faster and/or enhanced dissolving of the size range of granules of the relatively smaller TSG type may cause the granules of the relatively larger TSG type within the TSAP to be exposed. Further rotational motion of the TSAP over the treated subject's skin may exfoliate the subject's skin in and/or around the treated area. TSAP granules of the relatively larger TSG type (which dissolve relatively slower upon contact with the solvent during topical application) may: (1) dissolve slower and retain more of their original shape; (2) become exposed over the skin-facing surface of the TSAP as the granules of the relatively smaller TSG type, positioned between them, dissolve faster; and (3) collectively create a coarse (sand paper like) surface, enhancing the skin exfoliating properties of the rotating TSAP.

According to some embodiments, the TSAP application apparatus may include a vibrating electric motor/unit, powered by the power source and triggered/managed by the CPU/Control-Logic, for generating vibrational motion and transmitting the generated motion to the TSAP holder and the TSAP retained thereby, in order to facilitate the infusion of the therapeutic substances into the skin of the subject.

According to some embodiments, the generated vibrational motion may be in a substantially vertical direction to the surface of the treated subject's skin and may thus facilitate the infusion of the therapeutic substance(s) applied onto the treated skin area by repetitively pushing/driving them into the skin, wherein the skin area may already be exfoliated by the prior rotational motion of TSAP over it, further assisting the infusion process.

According to some embodiments, an apparatus for TSAP application may comprise a motor resistance level sensor functionally connected to a resistance-based control logic (connected to, or integrated into, the main CPU/Control-Logic), for respectively: measuring the resistance level applied on the spinning electric motor; and controlling the operation of the motor(s) and components/units of the apparatus based thereof.

According to some embodiments, one or more operation command(s), to the motor(s)/components/units/controllers of the TSAP application apparatus, may be triggered based on a motor resistance threshold value/pattern matching or crossing.

The vibrating motion, enhancing the infusion of the dissolved TSGs into the exfoliated skin, may accordingly, be automatically triggered based on the electric motor resistance, at a timing/stage within the topical application period of the TSAP, following to the dissolving of at least some of the TSGs (e.g. smaller granules) and the exfoliation of the skin in/around the treated area.

There may be provided a process by which to produce a TSAP according to embodiments of the present invention. The main stages of a TSAP Production Process may include: (1) combining a preselected ratio of amounts, of a first size range of TSG and a second, different, size range of TSG; (2) mixing the combined first sized TSG and a second sized TSG to create a substantially homogenous mixture of granules sizes; and (3) pressing the homogenous mixture in a mold forming a TSAP and removing the formed packet/tablet from the mold.

A process by which to produce a TSAP, in accordance with some embodiments, may include: (1) mixing one or more therapeutic active substance powder(s) with one or more non-therapeutic secondary substance powder(s) to a substantially homogeneous powder mix; (2) adding a fluid/gel/paste adhesive material (e.g. starch paste) to the mixture of powders; (3) mixing the fluid/gel/paste adhesive material (e.g. starch paste) with the mixture of powders; (4) drying the resulting mixture to form a solid lump; (5) milling the solid lump to form granules of various sizes; (6) stirring a lubricating material (e.g. solid fat) into the various sizes granules mixture; (7) using screen(s)/mesh(es) to filter/sieve the mixture into a first range of granule/sieve sizes and a second, larger, range of granule/sieve sizes; (8) mixing a preselected ratio of amounts of the first size range granules and the second size range granules; (9) pressing the granules mixture into a tablet/packet, wherein the resulting tablet/packet may consist of: second sized, larger, granules; having first sized, smaller, granules filling the spaces formed between them.

A process by which to produce a TSAP, in accordance with some embodiments, may include: (1) pressing a mixture of a first preselected ratio of amounts of a first size range of TSG and a second size range of TSG to form a first layer of a produced TSAP; and (2) pressing at least a second preselected ratio of amounts of a first size range of TSG and a second size range of TSG to form an additional layer of the produced TSAP. Some or all of the layers of the produced TSAP may, in accordance with some embodiments, include only a single size range of TSG.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1D show schematic views of the stages of composition and final resulting product of a Therapeutic Substance Application Packet (TSAP), in accordance with embodiments of the present invention;

FIG. 2A shows a first application stage—disposal—of a Therapeutic Substance Application Packet (TSAP), in accordance with embodiments of the present invention;

FIG. 2B shows a second application stage—exfoliation—of a Therapeutic Substance Application Packet (TSAP), in accordance with embodiments of the present invention;

FIG. 2C shows a third application stage—infusion—of a Therapeutic Substance Application Packet (TSAP), in accordance with embodiments of the present invention;

FIG. 3A shows an exemplary application apparatus of a Therapeutic Substance Application Packet (TSAP) including the main components and modules thereof, in accordance with embodiments of the present invention;

FIG. 3B shows an exemplary application apparatus of a Therapeutic Substance Application Packet (TSAP) including specific components and modules thereof (Heating Component, Vibrating Motor/Unit, Skin Resistance Level Sensor, Resistance Based Control Logic), in accordance with embodiments of the present invention;

FIG. 3C shows a flowchart of the main steps executed as part of an exemplary operation scheme of an application apparatus of a Therapeutic Substance Application Packet (TSAP), in accordance with some embodiments of the present invention;

FIG. 4A shows an exemplary Therapeutic Substance Application Packet (TSAP) Application Apparatus and a TSAP Holder thereof, wherein the TSAP Holder includes a TSAP retention mechanism, in accordance with embodiments of the present invention;

FIG. 4B is a cross section view of an exemplary Therapeutic Substance Application Packet (TSAP) Application Apparatus and a TSAP Holder thereof, wherein the TSAP Holder includes a TSAP retention mechanism, in accordance with embodiments of the present invention;

FIG. 4C is a cross section view showing in greater detail the retention mechanism of the Therapeutic Substance Application Packet (TSAP) Holder of FIG. 4B, in accordance with embodiments of the present invention;

FIG. 4D shows a flowchart of the steps executed by a Therapeutic Substance Application Packet (TSAP) Holder as part of an exemplary TSAP retention process, in accordance with embodiments of the present invention;

FIG. 5A shows a flowchart of the steps executed as part of an exemplary production process of a Therapeutic Substance Application Packet (TSAP), in accordance with embodiments of the present invention; and

FIG. 5B shows the main actions executed, and the resulting sub-products, of an exemplary production process of a Therapeutic Substance Application Packet (TSAP), in accordance with embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Unless specifically stated otherwise, as apparent from the following discussions, it is appreciated that throughout the specification discussions utilizing terms such as “processing”, “computing”, “calculating”, “determining”, or the like, may refer to the action and/or processes of a computer, computing system, computerized mobile device, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In addition, throughout the specification discussions utilizing terms such as “storing”, “hosting”, “caching”, “saving”, or the like, may be interchangeably used and may refer to the action and/or processes of ‘registering’, ‘recording’, ‘writing’ and/or ‘keeping’ digital information on a computer readable storage medium of a computer, computing system, or similar electronic computing device.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

Throughout the specification, discussions utilizing terms such as “Packet”, “Tablet”, “Pill”, “Pellet”, “Lozenge”, or the like, may refer to a solid form of any type or type combination of powder(s); and/or to granules/sieves of one or more powder types and/or one or more size ranges, compressed into solid form.

Functions, operations, components and/or features described herein with reference to one or more embodiments, may be combined with, or may be utilized in combination with, one or more other functions, operations, components and/or features described herein with reference to one or more other embodiments, or vice versa.

The present invention includes therapeutic compounds and mixtures, processes for producing same; and to systems, methods, devices, apparatuses circuits and computer executable code for the topical application of the therapeutic compounds and mixtures. According to some embodiments of the present invention, there may be provided a Therapeutic Substance Application Packet (TSAP), which packet may include a mixture of two or more types of therapeutic substance granules (TSG), wherein granules of a first TSG type may be of a different size than granules of a second TSG type. According to some embodiments. Granules of the first TSG type may be composed of the same or similar composition of therapeutic substances as are granules of the second TSG type. Alternatively, granules of the first TSG type may be composed of different compositions of therapeutic substances from granules of the second TSG type. A TSAP according to embodiments may include a preselected ratio of first type to second type TSGs. A size range of granules of the relatively smaller TSG type may be selected such as to dissolve relatively faster upon contact with a solvent during topical application, while a size range of granules of the relatively larger TSG type may be selected to dissolve relatively slower upon contact with a solvent during topical application and to retain exfoliating properties for at least a portion of a topical application period of the TSAP.

According to some embodiments of the present invention, a TSG may be composed of one or more medical, therapeutic, nutritional and/or cosmetic substances and/or active therapeutic substance types, including: (1) antioxidants; (2) hair growth stimulants; (3) hair removal substances; (4) skin nourishing and moisturizing substances; (5) skin protection substances; and/or (6) any combination of the above, optionally including the addition of one or more complementary substance(s), of the following substances: Vitamin A, Vitamin D, Vitamin E, Vitamin K, Vitamin C, Thiamin, Riboflavin, Niacin, Vitamin B6, Vitamin B12, Pantothenic Acid, Folic Acid, Biotin, Hyaluronic acid, Coenzyme Q10, Idebenone, Zinc, Copper, Beta carotene, Green tea, Pomegranates, Coffee berries, Grape seeds, Saw palmetto, Epilobium, Retinol, Peptides, Alpha and Beta hydroxy acids, Azelaic acid, Ursolic acid, Oleanolic acid and/or 4-Butyl Resorcinol.

According to some embodiments, the one or more TSG substance(s) may chemically react and/or dissolve, upon contact with a solvent previously applied to a treated organ section (e.g. face skin) of a subject, on which the TSAP is being applied. The solvent may be water or oil based and may take the form of: a gel, an oil, a cream or ointment, a lotion and/or a liquid. The solvent may be applied to the treated organ (e.g. face skin) of the subject, prior to the application of the TSAP and TSGs thereof, such that upon application one or more of the TSGs chemically reacts with the applied solvent, at least partially dissolving the granules of the TSG(s) to yield an infusible therapeutic fluid substance.

According to some embodiments of the present invention, a TSAP may be made to take any, or any combination, of the following shapes or forms: (1) a tablet shape in the form of a flat sided slice of a cylinder; (2) a disk shape in the form of a thin slice of a cylinder; (3) a spherical shape of a ball; (4) a tablet shape having bloated/swollen/convex side(s); (5) a polygon shape (e.g. a hexagon, an octagon); and/or (6) a cone shape.

According to some embodiments of the present invention, a TSAP may include two or more granules size ranges (TSG sizes), wherein the arrangement of the differently sized granules within the volume of the TSAP may be made to take any, or any combination, of the following TSGs arrangements: (1) Differently sized TSGs homogenously/evenly mixed over the entire volume of the TSAP; (2) Differently sized TSGs homogenously/evenly mixed within a partial section/space of the volume of the TSAP, with either only small TSGs, or only large TSGs, within the remaining volume; (3) Differently sized TSGs homogenously/evenly mixed within a partial section/space of the volume of the TSAP, only small TSGs within another partial section/space of the volume of the TSAP and only large TSGs within the remaining section/space of the volume of the TSAP; (4) a first layer of the TSAP consisting of a first size range of TSGs and another/other layer(s) of the TSAP consisting of a second size range of TSGs; and/or (5) an outer layer of the TSAP consisting of small sized TSGs and an inner section/space of the TSAP consisting of large sized TSGs, or vice versa.

According to some embodiments of the present invention, a TSAP may include, in addition to the two or more TSGs, any combination of the following substances, elements, materials and/or compounds: (1) a Catalyst for increasing the rate of a chemical reaction without itself undergoing any permanent chemical change; (2) a Binder/Adhesive for connecting between two or more substance type powders (e.g. an active substance powder and a secondary substance powder) to form a solid limp which is later milled and granulated to form the TSGs; (3) a Preservative for preserving the qualities of the TSGs' active substances (e.g. antioxidants) and extending the time period during which they remain effective; (4) a Lubricant for minimizing friction and separating TSGs while they are sieved into different size ranges; (5) Breakable microcapsules of solvent, or microencapsulated solvent, for breaking, during TSAP application, and releasing their internally (i.e. within capsule) contained solvents to dissolve the TSG(s) and collectively yield/form a therapeutic liquid; (6) Desiccant/Dehumidifying substances for acting as a TSG(s) drying agent (e.g. hygroscopic substances) used to prevent moisture from: contacting, reacting with and/or decreasing the effectiveness of, the TSG(s); (7) Inert abrasive/exfoliant for exfoliating the skin of a TSAP treated organ/area of a subject (e.g. face skin), thus facilitating the infusion of dissolved TSG(s) into the skin, wherein the inert attribute prevents the abrasive/exfoliant from chemically reacting with its surrounding substances (e.g. other TSG(s), solvent) thus retaining its structure and respective exfoliating characteristics; and/or (8) Any other type of Additive.

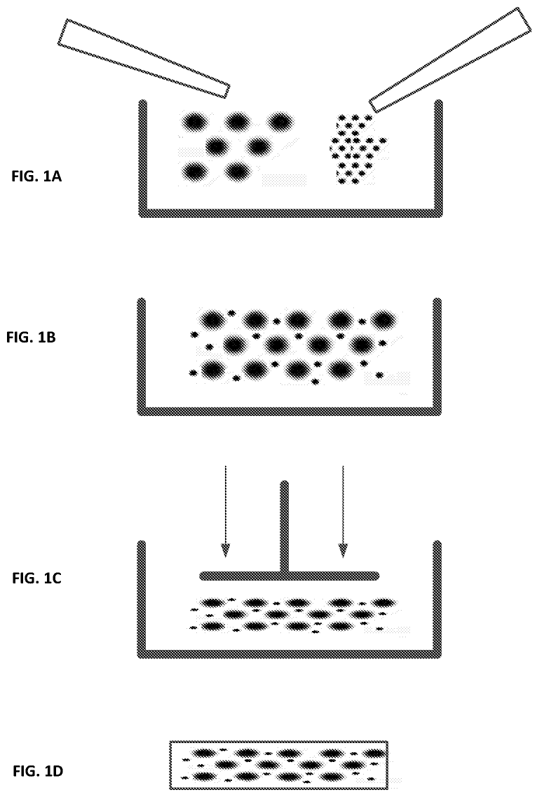

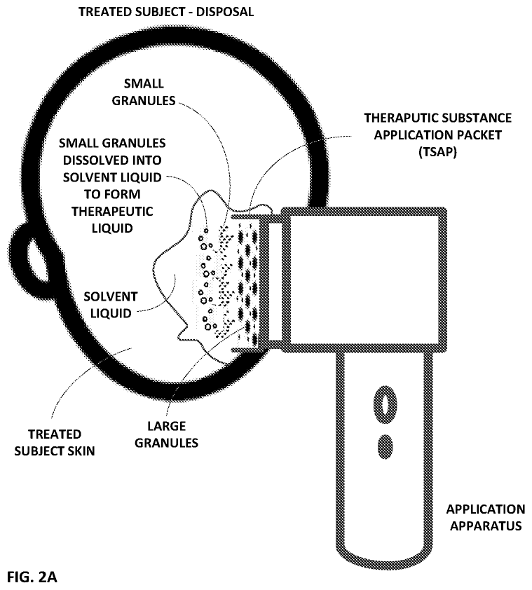

FIGS. 1A-1D show schematic views of the main stages of composition and final resulting product of a Therapeutic Substance Application Packet (TSAP), in accordance with embodiments of the present invention. In the figure, the following stages of composition are shown: FIG. 1A shows the mixing of two types of therapeutic substance granules (TSG), wherein granules of a first TSG type are of a different size than granules of a second TSG type; FIG. 1B shows a substantially homogeneous mixture including granules of a first TSG type and granules of a second TSG type having a different size/size-range; FIG. 1C shows the substantially homogeneous mixture being pressed into a mold; and FIG. 1D shows the resulting pressed TSAP, removed from the mold.

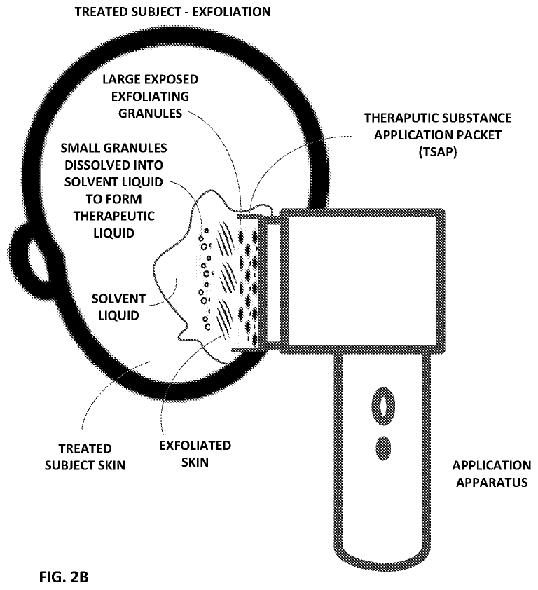

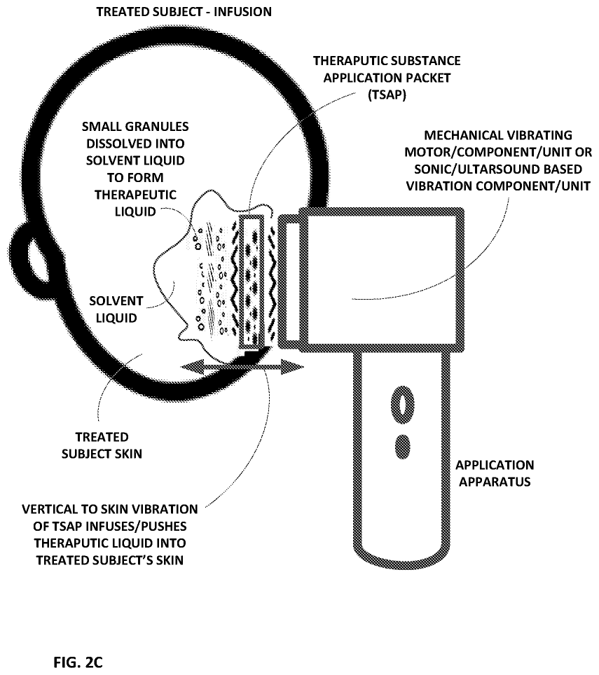

FIGS. 2A-2C show the main application stages—disposal, exfoliation and infusion—of a Therapeutic Substance Application Packet (TSAP), in accordance with embodiments of the present invention.

In FIG. 2A there is shown a disposal stage of a Therapeutic Substance Application Packet (TSAP), in accordance with embodiments of the present invention. In the figure, smaller sized granules of the first TSG type, dissolve into a solvent liquid, previously applied to the treated area of the treated subject's skin, to form a therapeutic liquid.

In FIG. 2B there is shown an exfoliation stage of a Therapeutic Substance Application Packet (TSAP), in accordance with embodiments of the present invention. In the figure, larger sized granules of the second TSG type, having exfoliating properties, are shown be exposed as a result of the faster dissolving of the smaller sized granulates positioned between them. In the figure, the skin of the subject at the treated area, coming in contact with the large exfoliating exposed granules, is shown to be exfoliated.

In FIG. 2C there is shown an infusion stage of a Therapeutic Substance Application Packet (TSAP), in accordance with embodiments of the present invention. In the figure, the dissolved small granules based therapeutic liquid, positioned over and/or around the now exfoliated area of the subject's skin, is pushed/infused towards/into the exfoliated skin area at least partially by the action of the shown mechanical vibrating motor/unit or sonic/ultrasound based vibration component/unit of the TSAP application apparatus, generating vertical to skin vibrations of the TSAP.

According to embodiments of the present invention, there may be provided a TSAP Application Apparatus to receive a TSAP and to apply its contents to the skin of a treated subject. The apparatus may include any combination of the following components: (1) an Electric Power Source (e.g. a rechargeable battery) for providing electric power to digital and mechanical components of the apparatus; (2) a TSAP Holder for receiving and connecting a TSAP to the apparatus, retaining it in position during use/operation and facilitating the application of the therapeutic substances thereof; (3) an Electric Motor, powered by the power source, for generating rotational motion and transmitting the generated motion to the TSAP holder and the TSAP retained thereby; and (4) a CPU/Control-Logic for triggering and managing the operation of the electric motor and thereby facilitating the application of the therapeutic substance(s) contained in the retained TSAP onto the surface of the skin of a treated subject and/or exfoliating the skin in and/or around the treated area to assist the infusion of therapeutic substance(s).

According to some embodiments, the different components of the TSAP application apparatus may be functionally enclosed in a body/casing; according to some embodiments, the body/casing may include a handle section connected, optionally in a right or approximately right angle, to an application and TSAP holding section, such that when a user of the apparatus holds the apparatus by the handle section, the outer surface of a TSAP retained by the TSAP holder is in a substantially horizontal plane to the surface of the treated subject's skin. According to some embodiments, the body/casing may constitute the handle, or holding area, of the TSAP application apparatus.

According to some embodiments, the TSAP application apparatus may further include a control interface, having user accessible interface elements, for powering the apparatus and for operating some or all of the motors/components/unit/controllers/functionalities of the apparatus as described herein.

According to some embodiments, the rotational motion of the TSAP holder and the TSAP retained thereby, may be in a substantially horizontal plane to the surface of the subject's skin, to enhance the application and the dissolving of the therapeutic substances onto the surface of the skin of the subject, by increasing the amount of contact between the TSAP granules and a solvent previously applied to the treated skin area of the subject.

According to some embodiments, the rotational motion of the TSAP over the treated subject's skin may, or may mostly, enhance the application and dissolving of the size range of granules of the relatively smaller TSG type, which dissolve relatively faster upon contact with a solvent during topical application.

According to some embodiments, the rotational motion of the TSAP holder and the TSAP retained thereby, may be in a substantially horizontal plane to the surface of the subject's skin, to exfoliate, or enhance the exfoliation, of the subject's skin and facilitate the absorption of the therapeutic substances.

According to some embodiments, the faster and/or enhanced dissolving of the size range of granules of the relatively smaller TSG type may cause the granules of the relatively larger TSG type within the TSAP to be exposed. Further rotational motion of the TSAP over the treated subject's skin may exfoliate the subject's skin in and/or around the treated area. TSAP granules of the relatively larger TSG type (which dissolve relatively slower upon contact with the solvent during topical application) may: (1) dissolve slower and retain more of their original shape; (2) become exposed over the skin-facing surface of the TSAP as the granules of the relatively smaller TSG type, positioned between them, dissolve faster; and (3) collectively create a coarse (sand paper like) surface, enhancing the skin exfoliating properties of the rotating TSAP.

According to some embodiments, the TSAP application apparatus may include a vibrating electric motor/unit, powered by the power source and triggered/managed by the CPU/Control-Logic, for generating vibrational motion and transmitting the generated motion to the TSAP holder and the TSAP retained thereby, in order to facilitate the infusion of the therapeutic substances into the skin of the subject.

According to some embodiments, the generated vibrational motion may be in a substantially vertical direction to the surface of the treated subject's skin and may thus facilitate the infusion of the therapeutic substance(s) applied onto the treated skin area by repetitively pushing/driving them into the skin, wherein the skin area may already be exfoliated by the prior rotational motion of TSAP over it, further assisting the infusion process.

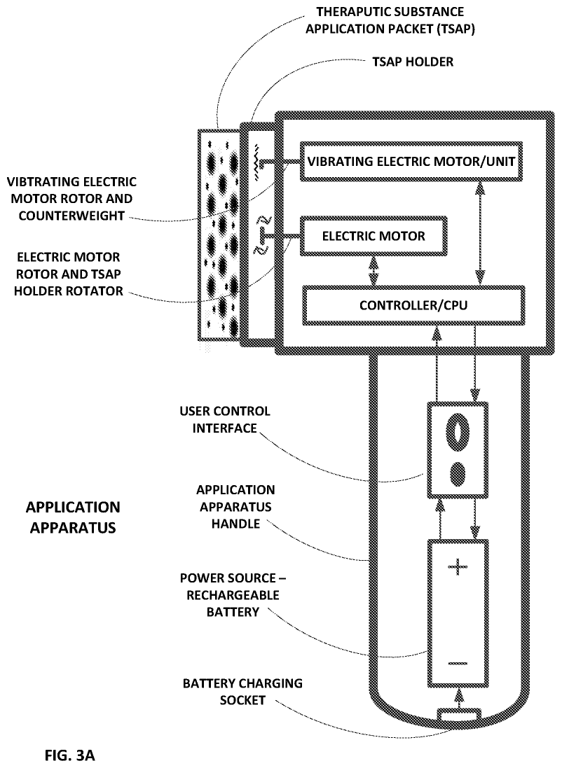

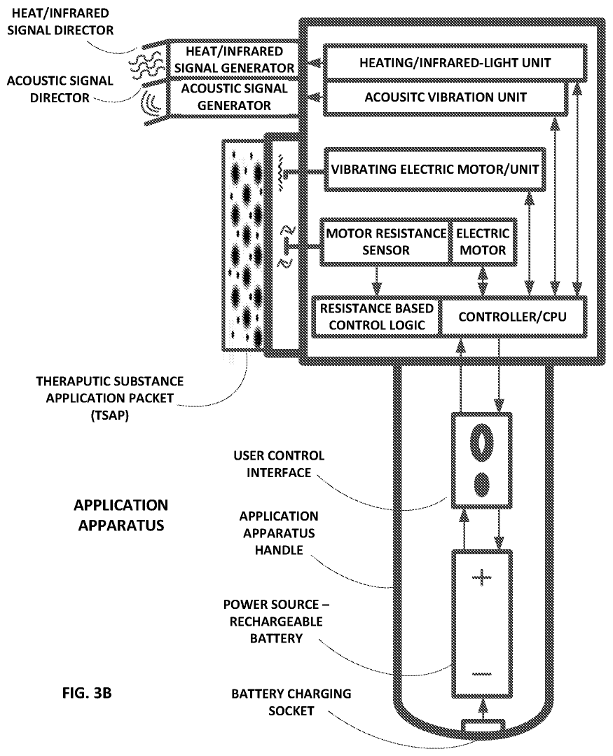

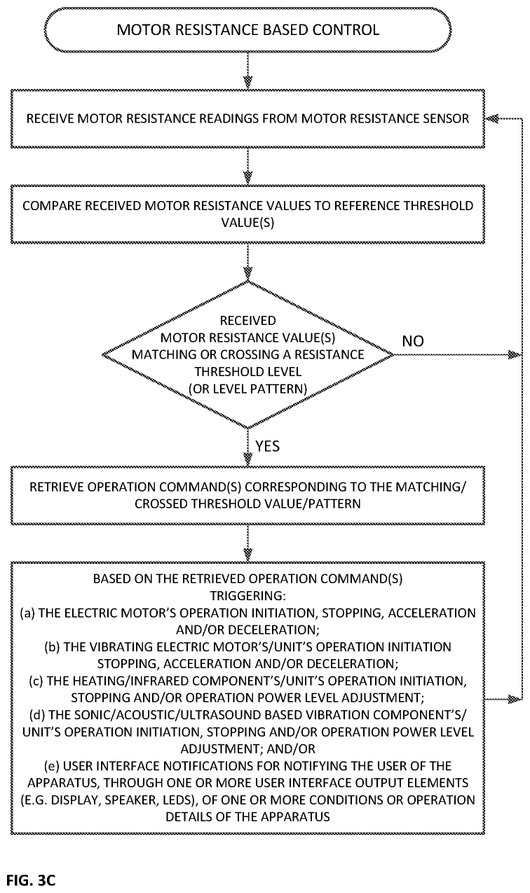

In FIG. 3A there is shown, an exemplary application apparatus of a Therapeutic Substance Application Packet (TSAP), in accordance with embodiments of the present invention. The shown apparatus includes: (1) a power source in the form of a rechargeable battery and a battery charging socket/circuits thereof; and (2) a user control interface for turning the apparatus ON and OFF and for controlling the operation of the apparatus. The shown user control interface is electrically connected to the rechargeable battery and functionally connected to a controller/CPU of the apparatus. In this example, the power source and the user control interface are contained-in/included-in/integrated-into the handle of the apparatus.

The shown apparatus further includes: (3) the mentioned controller/CPU for triggering and controlling the operation of each of the different modules and components of the apparatus and/or for managing the mutual/synchronized operation of the different modules and components of the apparatus in concert; (4) a TSAP holder for accepting and retaining a TSAP during its application process using the apparatus; (5) an electric motor, powered by the rechargeable battery, for generating rotational motion and transmitting it by utilizing the ‘electric motor rotor and TSAP holder rotator’ to the TSAP holder and its retained TSAP causing the TSAP to rotate/spin; and (6) a vibrating electric motor/unit, powered by the rechargeable battery, for generating vibrational motion and transmitting it by utilizing the ‘vibrating electric motor rotor and counterweight’ to the TSAP holder and its retained TSAP, causing the TSAP to vibrate.

According to some embodiments, an apparatus for TSAP application may comprise a heating/infrared component/unit, for generating heat/light/infra-red-light waves/signals and directing the signals to the treated area of the subject's skin, wherein the waves/signals enhance blood circulation at or around the treated skin area of the subject, to improve the infusion of the therapeutic substances.

According to some embodiments, an apparatus for TSAP application may comprise a sonic/acoustic/ultrasound based vibration component/unit, for generating sonic/acoustic/ultrasound signals and directing the signals to the treated area of the subject's skin, wherein the signals hitting the skin at or around the treated area may cause a vibratory motion of the skin. Vibrated skin may enhance blood circulation at or around the treated area, thus improving the Infusion of the therapeutic substances.

According to some embodiments, an apparatus for TSAP application may comprise a motor resistance level sensor functionally connected to a resistance-based control logic (connected to, or integrated into, the main CPU/Control-Logic), for respectively: measuring the resistance level applied on the spinning electric motor; and controlling the operation of the motor(s) and components/units of the apparatus based thereof.

According to some embodiments, the motor resistance level may, for example, be calculated and utilized as follows: (1) the resistance level sensor intermittently measures the rotational speed of the motor (e.g. in rounds per minute units), during its operation; (2) the resistance-based control logic calculates the ratio between: (a) the rotational speeds measured and (b) a known constant representing the motor speed operating with no external resistance applied (the greater [closer to 1] the calculated ratio is, the less resistance the motor is experiencing); (3) the resistance-based control logic compares: (a) the calculated ratios, or normalizations/derivations thereof, to (b) a reference threshold table including one or more motor resistance threshold level values (or resistance level patterns) and corresponding operation command(s) to the motor(s)/components/units/controllers of the apparatus; and (4) upon a calculated ratio matching, or crossing, a resistance threshold level (or level pattern) the corresponding operation command(s) may be triggered and/or matching operation commands/requests may be relayed to the main CPU/Control-Logic.

According to some embodiments, one or more of the following operation command(s), to the motor(s)/components/units/controllers of the TSAP application apparatus, may be triggered based on a motor resistance threshold value/pattern matching or crossing: (1) electric motor's operation initiation, stopping, acceleration and/or deceleration; (2) vibrating electric motor's/unit's operation initiation stopping, acceleration and/or deceleration; (3) heating/infrared component's/unit's operation initiation, stopping and/or operation power level adjustment; (4) sonic/acoustic/ultrasound based vibration component's/unit's operation initiation, stopping and/or operation power level adjustment; and/or (5) triggering of user interface notifications for notifying the user of the apparatus, through one or more user interface output elements (e.g. display, speaker, LEDs), of one or more conditions or operation details of the apparatus, for example: battery power status, engaged motors/components/units, user operation guidelines (e.g. push the apparatus and retained TSAP against skin with more/less force applied, wherein a green LED blinking on the user interface indicates more force should be applied and a red LED blinking indicates less force should be applied).

According to some embodiments, the sources of resistance experienced by the electric motor during its operation may, for example, be associated with: (1) the level of elasticity of the skin at or around the treated area; (2) the force applied on the apparatus and against the skin by the user; and/or (3) the operation stage of the apparatus, the operation status of any of its motors/components/units and/or the level to which the differently sized TSGs constituting the TSAP have dissolved.

According to some embodiments, an exemplary operation scenario of a TSAP application apparatus may include the utilization of the following apparatus components for execution of the respectively described steps: (1) The electric motor rotates the TSAP holder and the TSAP retained thereby against a skin area, of the treated subject, to which dissolvent has been previously applied; (2) TSAP granules of the relatively smaller TSG type dissolve faster than TSAP granules of the relatively larger TSG type, thereby exposing the larger granules' shapes and leading to a coarser/grainier TSAP application surface; (3) the now coarser/grainier TSAP application surface exfoliates the subject's skin at/around the treated area, while generating more friction over the skin and thus more resistance the rotational motion of the electric motor; (4) the motor resistance level sensor and functionally connected resistance-based control logic monitor the increasing motor resistance levels; upon the resistance level matching or crossing a reference resistance level threshold, the operation of the vibrating electric motor/unit is triggered.

The vibrating motion, enhancing the infusion of the dissolved TSGs into the exfoliated skin, may accordingly, be automatically triggered based on the electric motor resistance, at a timing/stage within the topical application period of the TSAP, following to the dissolving of at least some of the TSGs (e.g. smaller granules) and the exfoliation of the skin in/around the treated area.

In FIG. 3B there is shown, an exemplary application apparatus of a Therapeutic Substance Application Packet (TSAP), in accordance with embodiments of the present invention. The shown apparatus includes, additional specific components and modules over the basic components and modules of the apparatus shown in FIG. 3A. Additional components/modules shown, include: (1) a heat/infrared signal generator having a functionally associated heating/infrared-light unit/control-unit and a functionally associated heat/infrared signal director, wherein the generator, based on requests/commands from the heating/infrared-light unit/control-unit, generates heat/infrared signals which are directed by the heat/infrared signal director towards the treated area of the subject's skin. The heat/infrared signals hitting the treated skin area may cause it to heat thus facilitating the infusion/absorption of the therapeutic substance(s)/material(s) into it.

The exemplary application apparatus of a Therapeutic Substance Application Packet (TSAP) of FIG. 3B further includes: (2) an acoustic signal generator having a functionally associated acoustic vibration unit and a functionally associated acoustic signal director, wherein the generator, based on requests/commands from the acoustic vibration unit, generates acoustic signals which are directed by the acoustic signal director towards the treated area of the subject's skin. The acoustic signals hitting the treated skin area may cause it to vibrate thus facilitating the infusion/absorption of the therapeutic substance(s)/material(s) into it.

The exemplary application apparatus of a Therapeutic Substance Application Packet (TSAP) of FIG. 3B further includes: (3) a resistance based control logic having a functionally associated motor resistance sensor and further functionally associated with the controller/CPU. The resistance based control logic may relay to the controller/CPU operation requests/commands to the components of the apparatus, wherein the requests/commands and/or their time of triggering, are at least partially based on motor resistance level readouts received from the motor resistance sensor.

In FIG. 3C there is shown, a flowchart of the main steps/stages of an exemplary operation scheme of an application apparatus of a Therapeutic Substance Application Packet (TSAP), in accordance with some embodiments of the present invention. The shown steps include: (1) receiving motor resistance readings from motor resistance sensor; and (2) comparing received motor resistance values to reference threshold value(s).

If received motor resistance value(s) match or cross a resistance threshold level (or level pattern); then: (3) retrieving operation command(s) corresponding to the matching/crossed threshold value(s)/pattern(s); and (4) based on the retrieved operation command(s) triggering: (a) the electric motor's operation initiation, stopping, acceleration and/or deceleration; (b) the vibrating electric motor's/unit's operation initiation stopping, acceleration and/or deceleration; (c) the heating/infrared component's/unit's operation initiation, stopping and/or operation power level adjustment; (d) the sonic/acoustic/ultrasound based vibration component's/unit's operation initiation, stopping and/or operation power level adjustment; and/or (e) user interface notifications for notifying the user of the apparatus, through one or more user interface output elements (e.g. display, speaker, LEDs), of one or more conditions or operation details of the apparatus.

A TSAP Holder of a TSAP application apparatus, in accordance with embodiments of the present invention, may include a TSAP retention mechanism for accepting a TSAP and retaining it in position during application. A TSAP Retention Mechanism may include a Soft Material TSAP Holder for accepting a TSAP into a complementing slot, wherein at least part of the edge of the slot of the Soft Material TSAP Holder has an undercut angle cut out along it, thus creating a sharply angled slot edge, such that a TSAP pushed into the slot causes the soft and sharply angled material above the undercut angle to bend and/or squeeze to accommodate the TSAP. As the bent and/or squeezed soft and sharply angled material is biased to return to its original shape—it applies pressure against side edges of TSAP retaining it in position through use of the TSAP application apparatus.

According to some embodiments, the sharply angled slot edge of the retention mechanism may bend and/or squeeze to accommodate TSAPs of different sizes and/or shapes

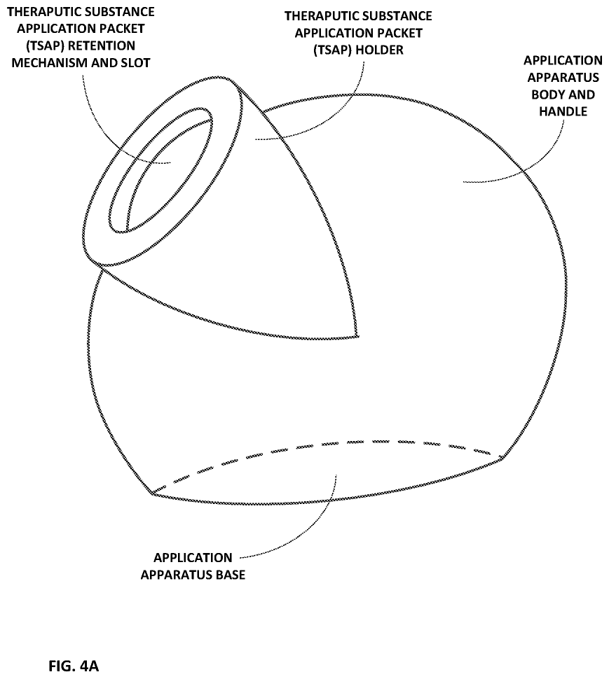

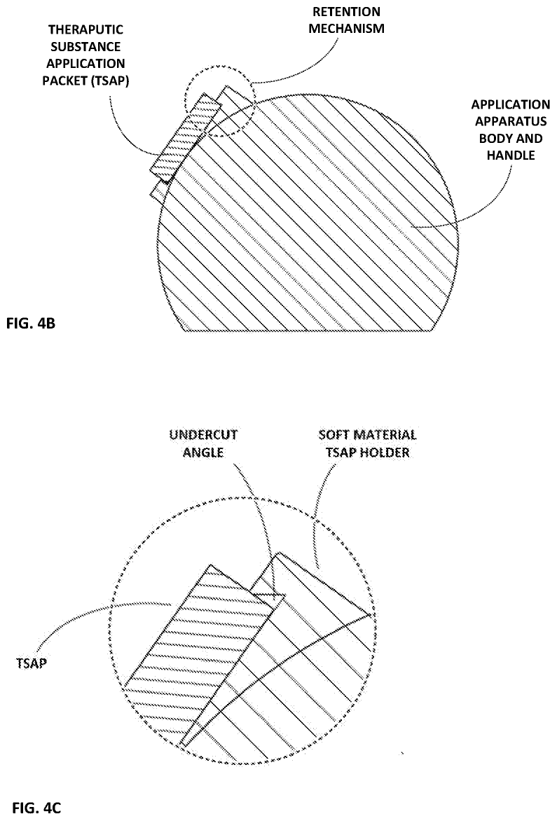

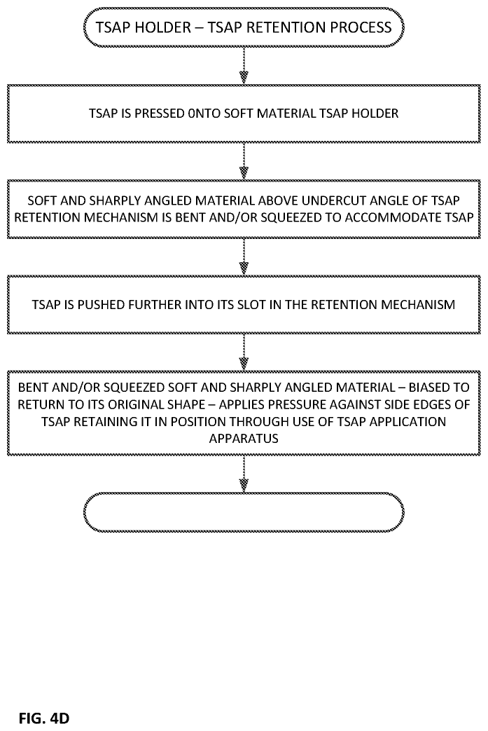

In FIG. 4A there is shown, an exemplary Therapeutic Substance Application Packet (TSAP) Application Apparatus and a TSAP Holder thereof, wherein the TSAP Holder includes a TSAP Retention Mechanism, in accordance with embodiments of the present invention. In the figure an oval/elliptic shaped body/casing of the exemplary application apparatus is shown. The body/casing may be utilized as a holding area or handle section and may house the internal components of the apparatus, such as, but not limited to: motors, controllers, power source, logics, units, sensors and the like. Further shown is the substantially flat base of the application apparatus that may be utilized for stably positioning the apparatus on a flat surface.

The TSAP Holder shown, is of a substantially conical shape, wherein the tip of the cone structure ‘penetrates’ into the oval/elliptic shaped body/casing of the application apparatus and is thus structurally and functionally integrated therewith. The outward facing base of the cone structure includes a TSAP Retention Mechanism and TSAP Slot thereof for accepting a TSAP and retaining it during its application to a subject by the apparatus.

In FIG. 4B there is shown, a cross section view of an exemplary Therapeutic Substance Application Packet (TSAP) Application Apparatus and a TSAP Holder thereof, wherein the TSAP Holder includes a TSAP retention mechanism and a TSAP retained thereby, in accordance with embodiments of the present invention.

In FIG. 4C there is shown, a cross section view showing in greater detail the retention mechanism of the Therapeutic Substance Application Packet (TSAP) Holder of FIG. 4B, in accordance with embodiments of the present invention. Shown retention mechanism is composed of a Soft Material TSAP Holder, wherein the soft material is adapted to at least partially bend or squeeze when pressure is applied against it—for example, by a TSAP being pressed into its slot/opening. The cross section of the edge of the slot/opening has an undercut edge, leaving/creating a sharply angled slot edge which is: more easily bent and/or squeezed to accommodate the TSAP; once bent/squeezed pushes back against the edges of the TSAP retaining it in position; and/or, enables for the insertion and retention of TSAPs of various sizes.

In FIG. 4D there is shown, a flowchart of the steps executed by a Retention Mechanism of a Therapeutic Substance Application Packet (TSAP) Holder as part of an exemplary TSAP retention process, in accordance with embodiments of the present invention. Shown steps include: (1) TSAP is pressed onto soft material TSAP holder; (2) soft and sharply angled material above undercut angle of TSAP retention mechanism is bent and/or squeezed to accommodate TSAP; (3) TSAP is pushed further into its slot in the retention mechanism; and/or (4) bent and/or squeezed soft and sharply angled material—biased to return to its original shape—applies pressure against side edges of TSAP retaining it in position through use of the TSAP application apparatus.

There may be provided a process by which to produce a TSAP according to embodiments of the present invention. The main stages of a TSAP Production Process may include: (1) combining a preselected ratio of amounts, of a first size range of TSG and a second, different, size range of TSG; (2) mixing the combined first sized TSG and a second sized TSG to create a substantially homogenous mixture of granules sizes; and (3) pressing the homogenous mixture in a mold forming a TSAP and removing the formed packet/tablet from the mold.

A process by which to produce a TSAP, in accordance with some embodiments, may include: (1) mixing one or more therapeutic active substance powder(s) with one or more non-therapeutic secondary substance powder(s) to a substantially homogeneous powder mix; (2) adding a fluid/gel/paste adhesive material (e.g. starch paste) to the mixture of powders; (3) mixing the fluid/gel/paste adhesive material (e.g. starch paste) with the mixture of powders; (4) drying the resulting mixture to form a solid lump; (5) milling the solid lump to form granules of various sizes; (6) stirring a lubricating material (e.g. solid fat) into the various sizes granules mixture; (7) using screen(s)/mesh(es) to filter/sieve the mixture into a first range of granule/sieve sizes and a second, larger, range of granule/sieve sizes; (8) mixing a preselected ratio of amounts of the first size range granules and the second size range granules; (9) pressing the granules mixture into a tablet/packet, wherein the resulting tablet/packet may consist of: second sized, larger, granules; having first sized, smaller, granules filling the spaces formed between them.

A process by which to produce a TSAP, in accordance with some embodiments, may include: (1) pressing a mixture of a first preselected ratio of amounts of a first size range of TSG and a second size range of TSG to form a first layer of a produced TSAP; and (2) pressing at least a second preselected ratio of amounts of a first size range of TSG and a second size range of TSG to form an additional layer of the produced TSAP. Some or all of the layers of the produced TSAP may, in accordance with some embodiments, include only a single size range of TSG.

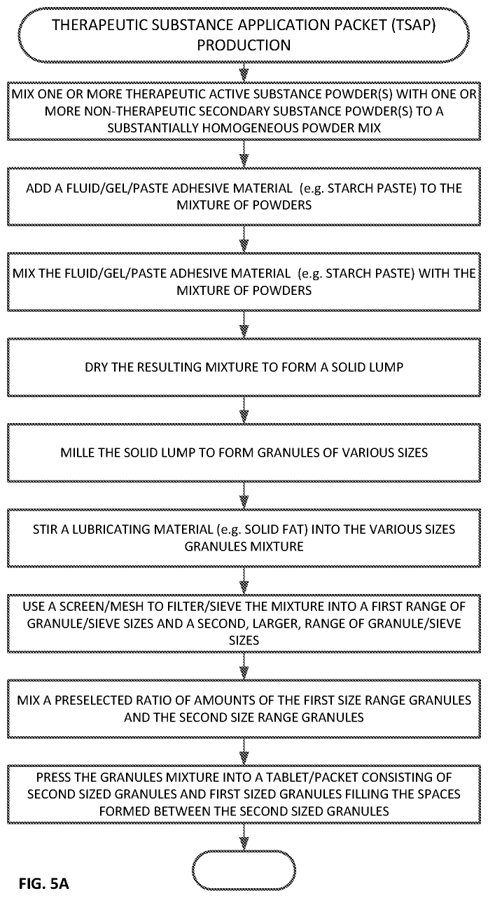

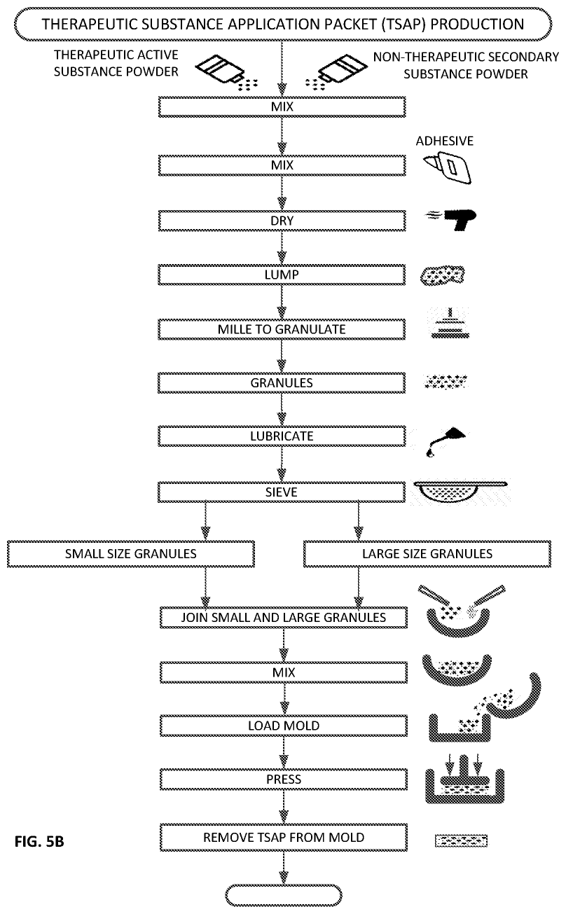

In FIG. 5A there is shown, a flowchart of the steps executed as part of an exemplary production process of a Therapeutic Substance Application Packet (TSAP), in accordance with embodiments of the present invention.

The shown process includes the following steps: (1) Mix one or more therapeutic active substance powder(s) with one or more non-therapeutic secondary substance powder(s) to a substantially homogenous powder mix; (2) Add a fluid/gel/paste adhesive material (e.g. starch paste) to the mixture of powders; (3) mix the fluid/gel/paste adhesive material (e.g. starch paste) with the mixture of powders; (4) dry the resulting mixture to form a solid lump; (5) mine the solid lump to form granules of various sizes; (6) stir a lubricating material (e.g. solid fat) into the various sizes granules mixture; (7) use a screen/mesh to filter/sieve the mixture into a first range of granule/sieve sizes and a second, larger, range of granule/sieve sizes; (8) mix a preselected ratio of amounts of the first size range granules and the second size range granules; and (9) press the granules mixture into a tablet/packet consisting of second sized granules and first sized granules filling the spaces formed between the second sized granules.

In FIG. 5B there is shown, a flowchart of the main actions executed, and the resulting sub-products, of an exemplary production process of a Therapeutic Substance Application Packet (TSAP), in accordance with embodiments of the present invention.

The shown process includes the following steps: (1) Mix—a therapeutic active substance powder and a non-therapeutic secondary substance powder; (2) Mix—an adhesive into the powders mixture; (3) Dry—the adhesive including powder mix; (4) Lump of powders mixture—formed after drying; (5) Mille—granulating the lump; (6) Granules—of different sizes formed; (7) Lubricate—the multi sized granules mix; (8) Sieve—the lubricated granules mix; (9) Granules size—sieving yields groups of granules, each including granules of a different size range; (10) Join—specific amounts of differently sized granules (e.g. based on a predetermined ratio of amounts); (11) Mix—the joined differently sized granules; (12) Load—mixture into a mold; (13) Press—the mold loaded mixture; and (14) Remove the mixture, pressed into a solid packet/tablet.

According to some embodiments of the present invention, a Therapeutic Substance Application Packet (TSAP) may include: and therapeutic substance granules (TSG) of a first size range; therapeutic substance granules (TSG) of a second, different, size range; wherein: (i) granules of the relatively smaller TSG size range, of the two TSGs size ranges, dissolve relatively faster upon contact with a solvent during topical application of the TSAP, (ii) granules of the relatively larger TSG size range, of the two TSGs size ranges, dissolve relatively slower upon contact with a solvent during topical application of the TSAP and retain exfoliating properties for at least a portion of a topical application period of the TSAP and (iii) the TSAP includes a preselected ratio of amounts between the TSG of the first size range and the TSG of the second size range.

According to some embodiments, the (TSG) of a first size range or the (TSG) of a second size range may include one or more therapeutic substances selected from the group consisting of: antioxidants, hair growth stimulants, hair removal substances, skin nourishing and moisturizing substances and skin protection substances.

According to some embodiments, the (TSG) of a first size range or the (TSG) of a second size range may include one or more complementary substances selected from the group consisting of: Vitamin A, Vitamin D, Vitamin E, Vitamin K, Vitamin C, Thiamin, Riboflavin, Niacin, Vitamin B6, Vitamin B12, Pantothenic Acid, Folic Acid, Biotin, Hyaluronic acid, Coenzyme Q10, Idebenone, Zinc, Copper, Beta carotene, Green tea, Pomegranates, Coffee berries, Grape seeds, Saw palmetto, Epilobium, Retinol, Peptides, Alpha and Beta hydroxy acids, Azelaic acid, Ursolic acid, Oleanolic acid and 4-Butyl Resorcinol.

According to some embodiments, the TSAP may include, in addition to the two or more TSGs, a substance combination selected from the group consisting of the following substance types: a Catalyst, a Binder/Adhesive, a Preservative, a Lubricant, Breakable microcapsules of solvent or microencapsulated solvent, Desiccant/Dehumidifying substances and Inert abrasive/exfoliant.

According to some embodiments, the TSG of a first size range or the TSG of a second size range chemically react and at least partially dissolve their granules—upon contact with a solvent previously applied to a treated organ section of a subject, on which the TSAP is being applied—to yield an infusible therapeutic fluid substance.

According to some embodiments, the shape or form of the TSAP may be selected from the group consisting of: a tablet shape in the form of a flat sided slice of a cylinder, a disk shape in the form of a thin slice of a cylinder, a spherical shape of a ball or ellipsoid, a tablet shape having bloated/swollen/convex side(s), a polygon shape and a cone or double cone shape.

According to some embodiments, the arrangement of the differently sized granules—of the (TSG) of a first size range and the (TSG) of a second size range—within the volume of the TSAP may be selected from the group of arrangements consisting of: Differently sized TSGs homogenously mixed over the entire volume of the TSAP, Differently sized TSGs homogenously mixed within a partial section of the volume of the TSAP with either only small TSGs or only large TSGs within the remaining volume, Differently sized TSGs homogenously mixed within a partial section of the volume of the TSAP only small TSGs within another partial section of the volume of the TSAP and only large TSGs within the remaining section of the volume of the TSAP, A first layer of the TSAP consisting of a first size range of TSGs and another layer(s) of the TSAP consisting of a second size range of TSGs and, An outer layer of the TSAP consisting of small sized TSGs and an inner section of the TSAP consisting of large sized TSGs or vice versa.

According to some embodiments of the present invention, a Therapeutic Substance Application Packet (TSAP) Application Apparatus may receive a TSAP and may apply its contents to the skin of a treated subject. The apparatus may include: an Electric Power Source for providing electric power; a TSAP Holder for receiving and connecting a TSAP to the apparatus and retaining it in position during its operation; an Electric Motor, powered by the power source, for generating rotational motion and transmitting the generated motion to the TSAP holder and the TSAP retained thereby, facilitating the disposal of the therapeutic substances onto the skin of the subject and exfoliating the skin of the subject at or around the treated area; and a Main Control Logic for managing the operation of the electric motor.

According to some embodiments, the TSAP Holder may include a TSAP Retention Mechanism comprising: a Soft Material TSAP Holder for accepting a TSAP into a complementing slot, wherein at least part of the edge of the slot of the Soft Material TSAP Holder has an undercut angle cut out along it, creating a sharply angled slot edge, such that a TSAP pushed into the slot causes the soft and sharply angled material above the undercut angle to bend or squeeze to accommodate the TSAP; and wherein the bent and/or squeezed soft and sharply angled material is biased to return to its original shape—applying pressure against side edges of the TSAP and retaining it in position through use of the TSAP application apparatus.

According to some embodiments, the TSAP Application Apparatus may include a Vibrating Electric Unit, powered by the power source, for generating vibrational motion and transmitting the generated motion to the TSAP holder and the TSAP retained thereby, thus facilitating the infusion of the therapeutic substances into the skin of the subject; wherein the Control-Logic may be adapted to manage the operation of the Vibrating Electric Unit and wherein managing at least partially includes automatically triggering the operation of the Vibrating Electric Unit while the Electric Motor is already operating, as part of the application of the TSAP.

According to some embodiments, the vibrational motion generated by the Vibrating Electric Unit may be in a substantially vertical direction to the surface of the treated skin of a subject using the TSAP Application Apparatus.

According to some embodiments, the TSAP Application Apparatus may include a Heating Unit for generating heat or light waves and directing them towards the surface of the treated skin of a subject using the TSAP Application Apparatus.

According to some embodiments, the TSAP Application Apparatus may include a Sonic Vibration Unit for generating acoustic signals and directing them towards the surface of the treated skin of a subject using the TSAP Application Apparatus.

According to some embodiments, the TSAP Application Apparatus may include a Motor Resistance Level Sensor for measuring the resistance level applied against the spinning of the Electric Motor and relaying resistance level indicative signals or data to a Resistance Based Control Logic functionally connected to, or integrated into, the Main Control Logic; wherein the Resistance Based Control Logic is adapted to manage the operation of the Electric Motor or the Vibrating Electric Unit at least partially based on the resistance level measured.

According to some embodiments, the Resistance Based Control Logic, as part of managing the operation of the Vibrating Electric Unit, is adapted to trigger the operation of the Vibrating Electric Unit upon receipt of signals or data indicative of the measured level of resistance matching or crossing a threshold resistance level value.

According to some embodiments, the Resistance Level Sensor may intermittently measure the rotational speed of the motor, during its operation; and the Resistance Based Control Logic may: (a) calculate the ratio between (i) the rotational speeds measured and (ii) a known constant representing the Electric Motor's rotational speed when operating with no external resistance applied, (b) compare (i) the calculated ratios, or normalizations or derivations thereof, to (ii) a reference threshold table including one or more motor resistance ratio threshold level values and corresponding ‘Vibrating Electric Unit operation command(s)’ and (c) upon a calculated ratio matching, or crossing, a resistance ratio threshold level—trigger the execution of the ‘Vibrating Electric Unit operation command(s)’ corresponding to the matched or crossed motor resistance ratio threshold level values.

According to some embodiments, the Resistance Based Control Logic, as part of managing the operation of the Vibrating Electric Unit, may be adapted to trigger the execution of one or more Vibrating Electric Unit operation command(s) based on the resistance level values measured; and operation command(s) to the Vibrating Electric Unit may be selected from the group consisting of: vibration initiation, vibration stopping, vibration speed acceleration and vibration speed deceleration.

According to some embodiments of the present invention, a method for producing a Therapeutic Substance Application Packet (TSAP), may include: milling a solid lump, of one or more therapeutic active substance powder(s) and one or more non-therapeutic secondary substance powder(s), to form a mixture of granules of various sizes; screening to filter or sieve the mixture into a first range of granule or sieve sizes and a second, larger, range of granule or sieve sizes; mixing a preselected ratio of amounts of the first size range granules and the second size range granules; and pressing the granules mixture into a tablet or packet, wherein the resulting tablet or packet consists of second sized, larger, granules—having first sized, smaller, granules filling the spaces formed between them.

According to some embodiments, the maximal size of first range of granule or sieve sizes may be less-than the minimal size of the second, larger, range of granule or sieve sizes.

The subject matter described above is provided by way of illustration only and should not be constructed as limiting. While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A Therapeutic Substance Application Packet (TSAP), said TSAP including:
therapeutic substance granules (TSG) of a first size range;
therapeutic substance granules (TSG) of a second, different, size range; and
wherein: (i) granules of the relatively smaller TSG size range, of said two TSGs size ranges, dissolve relatively faster upon contact with a solvent during topical application of said TSAP, (ii) granules of the relatively larger TSG size range, of said two TSGs size ranges, dissolve relatively slower upon contact with a solvent during topical application of said TSAP and retain exfoliating properties for at least a portion of a topical application period of said TSAP and (iii) said TSAP includes a preselected ratio of amounts between said TSG of the first size range and said TSG of the second size range.

2. The Therapeutic Substance Application Packet (TSAP) of claim 1, wherein said (TSG) of a first size range or said (TSG) of a second size range include one or more therapeutic substances selected from the group consisting of: antioxidants, hair growth stimulants, hair removal substances, skin nourishing and moisturizing substances and skin protection substances.

3. The Therapeutic Substance Application Packet (TSAP) of claim 2, wherein said (TSG) of a first size range or said (TSG) of a second size range further include one or more complementary substances selected from the group consisting of: Vitamin A, Vitamin D, Vitamin E, Vitamin K, Vitamin C, Thiamin, Riboflavin, Niacin, Vitamin B6, Vitamin B12, Pantothenic Acid, Folic Acid, Biotin, Hyaluronic acid, Coenzyme Q10, Idebenone, Zinc, Copper, Beta carotene, Green tea, Pomegranates, Coffee berries, Grape seeds, Saw palmetto, Epilobium, Retinol, Peptides, Alpha and Beta hydroxy acids, Azelaic acid, Ursolic acid, Oleanolic acid and 4-Butyl Resorcinol.

4. The Therapeutic Substance Application Packet (TSAP) of claim 3, wherein said TSAP further includes, in addition to the two or more TSGs, a substance combination selected from the group consisting of the following substance types: a Catalyst, a Binder/Adhesive, a Preservative, a Lubricant, Breakable microcapsules of solvent or microencapsulated solvent, Desiccant/Dehumidifying substances and Inert abrasive/exfoliant.

5. The Therapeutic Substance Application Packet (TSAP) of claim 1, wherein said TSG of a first size range or said TSG of a second size range chemically react and at least partially dissolve their granules—upon contact with a solvent previously applied to a treated organ section of a subject, on which the TSAP is being applied—to yield an infusible therapeutic fluid substance.

6. The Therapeutic Substance Application Packet (TSAP) of claim 1, wherein the shape or form of said TSAP is selected from the group consisting of: a tablet shape in the form of a flat sided slice of a cylinder, a disk shape in the form of a thin slice of a cylinder, a spherical shape of a ball or ellipsoid, a tablet shape having bloated/swollen/convex side(s), a polygon shape and a cone or double cone shape.

7. The Therapeutic Substance Application Packet (TSAP) of claim 1, wherein the arrangement of the differently sized granules—of said (TSG) of a first size range and said (TSG) of a second size range—within the volume of the TSAP is selected from the group of arrangements consisting of: Differently sized TSGs homogenously mixed over the entire volume of the TSAP, Differently sized TSGs homogenously mixed within a partial section of the volume of the TSAP with either only small TSGs or only large TSGs within the remaining volume, Differently sized TSGs homogenously mixed within a partial section of the volume of the TSAP only small TSGs within another partial section of the volume of the TSAP and only large TSGs within the remaining section of the volume of the TSAP, A first layer of the TSAP consisting of a first size range of TSGs and another layer(s) of the TSAP consisting of a second size range of TSGs and, An outer layer of the TSAP consisting of small sized TSGs and an inner section of the TSAP consisting of large sized TSGs or vice versa.

8. A Therapeutic Substance Application Packet (TSAP) Application Apparatus to receive a TSAP and to apply its contents to the skin of a treated subject, said apparatus including:
an Electric Power Source for providing electric power;
a TSAP Holder for receiving and connecting a TSAP to said apparatus and retaining it in position during its operation;
an Electric Motor, powered by said power source, for generating rotational motion and transmitting the generated motion to said TSAP holder and the TSAP retained thereby, facilitating the disposal of the therapeutic substances onto the skin of the subject and exfoliating the skin of the subject at or around the treated area; and
a Main Control Logic for managing the operation of said electric motor.

9. The TSAP Application Apparatus of claim 8, wherein said TSAP Holder includes a TSAP Retention Mechanism comprising:
a Soft Material TSAP Holder for accepting a TSAP into a complementing slot, wherein at least part of the edge of the slot of said Soft Material TSAP Holder has an undercut angle cut out along it, creating a sharply angled slot edge, such that a TSAP pushed into the slot causes the soft and sharply angled material above the undercut angle to bend or squeeze to accommodate the TSAP; and wherein the bent and/or squeezed soft and sharply angled material is biased to return to its original shape—applying pressure against side edges of the TSAP and retaining it in position through use of said TSAP application apparatus.

10. The TSAP Application Apparatus of claim 8, further including a Vibrating Electric Unit, powered by said power source, for generating vibrational motion and transmitting the generated motion to said TSAP holder and the TSAP retained thereby, thus facilitating the infusion of the therapeutic substances into the skin of the subject; wherein said Control-Logic is further adapted to manage the operation of said Vibrating Electric Unit and wherein managing at least partially includes automatically triggering the operation of said Vibrating Electric Unit while said Electric Motor is already operating, as part of the application of the TSAP.

11. The TSAP Application Apparatus of claim 10, wherein the vibrational motion generated by said Vibrating Electric Unit is in a substantially vertical direction to the surface of the treated skin of a subject using said TSAP Application Apparatus.

12. The TSAP Application Apparatus of claim 8, further including a Heating Unit for generating heat or light waves and directing them towards the surface of the treated skin of a subject using said TSAP Application Apparatus.

13. The TSAP Application Apparatus of claim 8, further including a Sonic Vibration Unit for generating acoustic signals and directing them towards the surface of the treated skin of a subject using said TSAP Application Apparatus.

14. The TSAP Application Apparatus of claim 10, further including a Motor Resistance Level Sensor for measuring the resistance level applied against the spinning of said Electric Motor and relaying resistance level indicative signals or data to a Resistance Based Control Logic functionally connected to, or integrated into, said Main Control Logic; wherein said Resistance Based Control Logic is adapted to manage the operation of said Electric Motor or said Vibrating Electric Unit at least partially based on the resistance level measured.

15. The TSAP Application Apparatus of claim 14, wherein said Resistance Based Control Logic, as part of managing the operation of said Vibrating Electric Unit, is adapted to trigger the operation of said Vibrating Electric Unit upon receipt of signals or data indicative of the measured level of resistance matching or crossing a threshold resistance level value.

16. The TSAP Application Apparatus of claim 14, wherein:
said Resistance Level Sensor intermittently measures the rotational speed of the motor, during its operation; and
said Resistance Based Control Logic:
(a) calculates the ratio between (i) the rotational speeds measured and (ii) a known constant representing said Electric Motor's rotational speed when operating with no external resistance applied, (b) compares (i) the calculated ratios, or normalizations or derivations thereof, to (ii) a reference threshold table including one or more motor resistance ratio threshold level values and corresponding ‘Vibrating Electric Unit operation command(s)’ and (c) upon a calculated ratio matching, or crossing, a resistance ratio threshold level—triggers the execution of the ‘Vibrating Electric Unit operation command(s)’ corresponding to the matched or crossed motor resistance ratio threshold level values.

17. The TSAP Application Apparatus of claim 14, wherein said Resistance Based Control Logic, as part of managing the operation of said Vibrating Electric Unit, is adapted to trigger the execution of one or more Vibrating Electric Unit operation command(s) based on the resistance level values measured; and wherein operation command(s) to said Vibrating Electric Unit are selected from the group consisting of: vibration initiation, vibration stopping, vibration speed acceleration and vibration speed deceleration.

18. A method for producing a Therapeutic Substance Application Packet (TSAP), said method including:
milling a solid lump, of one or more therapeutic active substance powder(s) and one or more non-therapeutic secondary substance powder(s), to form a mixture of granules of various sizes;
screening to filter or sieve the mixture into a first range of granule or sieve sizes and a second, larger, range of granule or sieve sizes;
mixing a preselected ratio of amounts of the first size range granules and the second size range granules; and
pressing the granules mixture into a tablet or packet, wherein the resulting tablet or packet consists of second sized, larger, granules—having first sized, smaller, granules filling the spaces formed between them.

19. The method according to claim 18, wherein the maximal size of first range of granule or sieve sizes is less-than the minimal size of the second, larger, range of granule or sieve sizes.
